(19) United States
(12) Patent Application Publication
(10) Pub. No.: US 2009/0082723 A1
(43) Pub. Date: Mar. 26, 2009

(54) Medical devices and methods for their fabrication and use

(76) Inventors: Magnus Krogh, Linkoping (SE); Edwin Jager, Linkoping (SE)

Correspondence Address: HARNESS, DICKEY & PIERCE, P.L.C., P.O. BOX 8910, RESTON, VA, 20195, US

(21) Appl. No.: 12/153,444

(22) Filed: Sep. 8, 2008

Related U.S. Application Data
(63) Continuation of application No. PCT/EP2006/010838, filed on Nov. 13, 2006.
(60) Provisional application No. 60/737,413, filed on Nov. 17, 2005.

(30) Foreign Application Priority Data
Nov. 17, 2005 (SE) 0502529-1

(51) Int. Cl. A61M 25/092

(52) U.S. Cl. 604/95.05

(57) ABSTRACT

An elongate device for introduction into a body lumen comprises a body extending in a longitudinal direction (L), and having at least two skeletal members (21, 21a, 21b, 24, 27a, 27b), which are substantially aligned in the longitudinal direction (L), and at least one electroactive polymer material (22), which changes volume upon electrical activation, arranged to control a distance between two longitudinally spaced-apart portions of said skeletal members (21, 21a, 21b, 24, 27a, 27b). The body presents an asymmetric bending stiffness, and/or the electroactive polymer material is asymmetrically arranged about a central axis of the device, such that the body is arranged to bend transversely of the longitudinal direction (L) upon activation of the electroactive polymer material. The electroactive polymer material (22) is form fit onto at least one of the skeletal members (21, 21a, 21b).

There is further provided an elongate device for introduction into a body lumen, the elongate device having a controllable stiffness portion.

Methods for fabricating and using the elongate devices are also provided.

### PRIORITY

This application is a continuation of PCT/EP2006/010838 filed on Nov. 13, 2006, which is an international application claiming priority from SE 0502529-1 filed Nov. 17, 2005 and U.S. 60/737,413 filed Nov. 17, 2005, the entire contents of which are hereby incorporated by reference.

### TECHNICAL FIELD

The invention relates to medical devices, and in particular to elongate devices for introduction into a body lumen, usable in e.g. catheters, guidewires and tools for vascular surgery, vascular intervention or endoscopy.

### BACKGROUND

In many areas of vascular surgery, guidewires, leads and catheters are used to reach specific areas inside the body, e.g. in the vascular system. These tools are generally passive, a condition that sometimes limits, or unnecessarily prolongs the procedure. Adding active steering capabilities to, for instance guidewires, would make it simpler to reach a desired area and thereby facilitate the procedure.

Also, the stiffness of the medical devices is generally fixed. During a surgical procedure it would be an advantage if the surgeon could change the stiffness of the medical device. In some instances the device should be stiff and more rigid in order to achieve a good pushability, and to be able to penetrate obstructions, and in other cases, the device should be flexible and soft to be able to follow curvatures and bends. At present, a trade-off between the two opposite demands has to be made. Controllable stiffness would reduce the trade-off significantly.

Electroactive polymers (EAP) are a novel class of materials that have electrically controllable properties. An overview on electroactive polymers can be found in “Electroactive Polymers (EAP) Actuators as Artificial Muscles—Reality, Potential, and Challenges” 2^{nd} ed. Y. Bar-Cohen (ed.) ISBN 0-8194-5297-1.

One class of EAPs are conducting polymers. These are polymers with a backbone of alternating single and double bonds. These materials are semiconductors and their conductivity can be altered from insulating to conducting with conductivities approaching those of metals. Polypyrrole (PPy) is one such conducting polymer and will be taken here as an example.

PPy can be electrochemically synthesised from a solution of pyrrole monomers and a salt as is know to those skilled in the art. After synthesis PPy is in its oxidised, or also called doped, state. The polymer is doped with an anion A-.

PPy can be electrochemically oxidised and reduced by applying the appropriate potential to the material. This oxidation and reduction is accompanied with the transport of ions and solvents into and out of the conductive polymer. This redox reaction changes the properties of polypyrrole, such as the conductivity, colour, modulus of elasticity and volume.

Two different schemes of redox are possible:

If PPy is doped with a large, immobile anion A- scheme 1 occurs, which schematically can be written as:

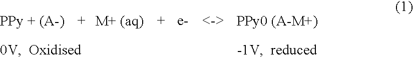

When PPy is reduced to its neutral state, cations M+ including their hydration shell and solvent are inserted into the material and the material swells. When PPy is oxidised again the opposite reaction occurs, M+ cations (including hydration shell and solvent) leave the material and its volume decreases.

If on the other hand PPy is doped with small, mobile anions a-, scheme 2 occurs:

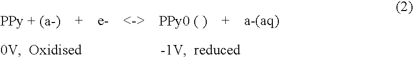

In this case the opposite behaviour of scheme 1 occurs. In the reduced state, the anions leave the material and it shrinks. The oxidised state is now the expanded state and the reduced state the contracted. Non limiting example of ions A- is dodecylbenzene sulfonate (DBS-), of a-perchlorate (ClO4-), and of M+ sodium (Na+) or lithium (Li+).

This volume change can for instance be used to build actuators (See Q. Pei and O. Inganäs, “Conjugated polymers and the bending cantilever method: electrical muscles and smart devices”, Advanced materials, 1992, 4(4), p. 277-278. and Jager et al., “Microfabricating Conjugated Polymer Actuators”, Science 2000 290: 1540-1545).

This redox reaction is usually driven in an electrochemical cell that comprises a working electrode (i.e. the conducting polymer) and counter electrode, preferably a reference electrode, and an electrolyte.

The electrolyte may be an aqueous salt solution, but can be a solid polymer electrolyte, a gel, a non-aqueous solvent, and ionic liquids as is know to those skilled in the art, but even biologically relevant environments such as blood (plasma), cell culture media, physiological media, ionic contrast solutions, etc can be used.

US2003/0236445 discloses a controllably bendable catheter using EAP actuators. However, the bending is generated by adding a plurality of complex multilayer EAP based linear actuators. The EAP actuators comprise an active member of EAP, an electrolyte and a counter electrode. All of these components are encapsulated to form an actuator. This requires both a complex fabrication of each single multilayer actuator (including unsolved issues such as encapsulation) and a cumbersome mounting of each individual actuator to the catheter/endoscope. Also, as each actuator is individually controlled, complex addressing and control schemes are needed.

In US2005/0165439, referring to FIGS. 9A-11B thereof, it is proposed to use an expanding ring of EAP in order to increase the bending stiffness according to the principle that the bending stiffness is proportional to D̂4 where the D is the outer diameter. Increasing the volume of an EAP ring is predicted to increase the diameter and thus the bending stiffness. However, the present inventors have discovered that the predictions made in US2005/0165439 regarding the relation between stiffness properties and change in outer diameter are not universally valid. Hence, the device disclosed in US2005/0165439 is unreliable, since it does not always operate as described therein.

### SUMMARY OF THE INVENTION

A general object is to provide an elongate device for introduction into the body lumen, which eliminates or at least alleviates the disadvantages of the prior art.

A first specific object is to provide a more simple, reliable and easy to manufacture controllable elongate device for introduction into a body lumen.

A second specific object is to provide an improved elongate device for introduction into a body lumenhaving a variable and controllable stiffness.

The objects are wholly or partially achieved by a devices, systems and methods as set forth in the respective independent claim. Embodiments are set forth in the attached dependent claims and in the following description and drawings.

Hence, there is provided an elongate device for introduction into a body lumen, the device comprising a body extending in a longitudinal direction (L), and having least two skeletal members, which are substantially aligned in a longitudinal direction of the device, and at least one electroactive polymer material which changes volume upon electrical activation, arranged to control a distance between two longitudinally spaced-apart portions of said skeletal members. The body presents an asymmetric bending stiffness, and/or the electroactive polymer material is asymmetrically arranged about a central axis of the device, such that the body is arranged to bend transversely of the longitudinal direction (L) upon activation of the electroactive polymer material. The electroactive polymer material is form fit onto an outside of at least one of the skeletal members.

By a “skeletal member” is understood a part which provides a skeleton structure or a frame of the device Hence, the skeletal member may be e.g. a discrete element, such as a disk or a ring, or a turn of a bendable helix. The skeletal members may be made from any material such as metal, polymer, composites thereof.

By “form-fit” is meant that the polymer material is e.g. synthesized, cast, applied or moulded into a shape corresponding to at least a part of the skeletal member. The definition is intended to cover both the instance in which the electroactive polymer material is synthesized, cast or molded directly onto the skeletal member, and the instance where the electroactive polymer material is first formed into a desired shape and subsequently assembled with the skeletal member.

By form-fitting the EAP material to the skeletal members, it is possible to utilize the volume changing properties of the EAP material without having to first manufacture an actuator, which is then mounted onto at least one of the skeletal members. This enables simplified and more reliable production of controllable elongate medical devices, such as catheters or endoscopes.

The electroactive polymer material may be formed directly onto said at least one of the skeletal members. This provides for simple and reliable forming of the electroactive polymer material, and may completely eliminate the need for mounting the electroactive polymer material to the skeletal members.

The at least one electroactive polymer material may extend over a portion of a cross section in a transverse direction of the device, which portion is smaller than the total area of the device cross section. This enables provision of a controllable device using a smaller amount of electroactive polymer material.

The device may further comprise a second electroactive polymer material arranged between said skeletal members to control a distance between another two longitudinally spaced-apart portions of said skeletal members. Thus, a stronger and/or more accurate device can be provided. This also enables a device that is controllable in different directions.

The at least one electroactive polymer material and the second electroactive polymer material may be electrically insulated from each other. Thus, the electroactive materials may be individually controllable.

One of the at least one electroactive polymer material and the second electroactive polymer material may comprise an electroactive polymer material that is expandable when subjected to an externally applied electrical signal, and another one of the at least one electroactive polymer material and the second electroactive polymer material comprises an electroactive polymer material that is contractable when subjected to the externally applied electrical signal. Thus, two electroactive material portions may be controlled using a single electrical signal applied to both of them simultaneously.

Also the second electroactive polymer material is form fit onto at least one of the skeletal members.

The at least one electroactive polymer material and the second electroactive polymer material may extend over a respective portion of a cross section in a transverse direction of the device, which portions are smaller than the total area of the cross section in the transverse direction of the device.

The device may further comprise a third electroactive polymer material arranged between said skeletal members to control a distance between yet another two longitudinally spaced-apart portions of said skeletal members, each of said at least one, said second and said third electroactive polymer material being individually controllable through respective externally applied electrical signals. Hence, a device which is controllable in several directions can be achieved.

In one embodiment, at least one of the skeletal members, has a varying thickness in a direction perpendicular to the longitudinal direction of the device.

In another embodiment, at least one of the skeletal members comprises two transversely juxtaposed portions having different modulus of elasticity.

In yet another embodiment, the at least two longitudinally juxtaposed skeletal members are integrally formed from a flexible material, and separated by a crease.

In yet another embodiment, the skeletal members form separate parts, which are arranged in a mutually longitudinally spaced relationship to form the device.

In yet another embodiment, the skeletal members are connected to each other forming substantially a helix.

In yet another embodiment, longitudinally spaced-apart portions of the skeletal members are connected to each other, by a material other than the electroactive polymer material.

In one embodiment, a material is arranged to cover at least a part of the skeletal members. The material may be insulating and an electrode may be arranged on the material.

The material may be arranged to substantially fill a cavity enclosed by the skeletal members. Such a cavity may be formed by a the skeletal members being of annular shape or turns of a coil.

A reinforcing core may be arranged in a longitudinal cavity enclosed by the skeletal members.

The reinforcing core may be conducting and provided with an ion conducting, electrically insulating covering.

An electrode may be arranged on the reinforcing core.

A reinforcing casing may be arranged to enclose the skeletal members. An electrode may be arranged on the reinforcing casing.

The casing may be insulating or ion conducting but electrically insulating.

The casing may be ion insulating, i.e. capable of enclosing an electrolyte. According to a second aspect, there is provided a method for providing an elongate device for introduction into a body lumen, comprising a body extending in a longitudinal direction, and having at least two skeletal members, which are substantially aligned in the longitudinal direction, and at least one electroactive polymer material, which changes volume upon electrical activation, arranged to control a distance between two longitudinally spaced-apart portions of said skeletal members. The body presents an asymmetric bending stiffness, and/or the electroactive polymer material is asymmetrically arranged about a central axis of the device, such that the body is arranged to bend transversely of the longitudinal direction upon activation of the electroactive polymer material. The method further comprises form-fitting the electroactive polymer material onto the skeletal members.

The electroactive polymer material may be formed directly onto at least one of the skeletal members.

In the method, a mask may be provided on such portions of the skeletal members that are not to be covered by the electroactive polymer material.

The method may comprise forming the electroactive polymer material directly onto at least one of the skeletal members, and removing only part of the electroactive polymer material between the skeletal members.

The method may comprise forming the electroactive polymer material directly onto at least one of the skeletal members, and passivating only part of the electroactive polymer material between the skeletal members.

Furthermore, there is provided an elongate device for introduction into a body lumen, the device comprising an elongate body extending in a longitudinal (L), and having a controllable stiffness portion, comprising an electroactive polymer material. The stiffness of the controllable stiffness portion is controllable by applying an electrical signal to the electroactive polymer material to change the modulus of elasticity of the electroactive polymer material.

By “controllable stiffness portion” is understood a portion of the device, whose stiffness can be altered, i.e. increased or decreased.

As mentioned above, this aspect is based on an insight that the prediction made in US2005/0165439 regarding the change in stiffness properties due to the increase in outer diameter is not universally valid.

Actually, electroactive polymers, such as conducting polymers, not only change their volume upon electrical stimulation, but also their material properties, such as the Young's (or elastic) modulus ‘E’. For instance the Young's modulus for PPy(DBS), polypyrrole doped with dodecylbenzene sulfonate is about 200 MPa in the reduced state and 500 MPa in the oxidised state, as is known per se from L. Bay, K. West, and S. Skaarup, “Pentanol as co-surfactant in polypyrrole actuators”, Polymer, 2002, 43(12), p. 3527-3532. This enables devices of which the material properties and thus mechanical properties such as stiffness can be altered actively. The stiffness of an elongate device is proportional to the product of E*I. For tubular devices, the moment of inertia I contains a proportionality D̂4, where D is the diameter. So, as an EAP ring according to Scheme 1 such as PPy(DBS) increases its thickness, the material shift from its oxidised to its reduced state and thus the Young's modulus decreases. The product of E*D̂4 decreases and the device becomes more flexible. This principle applies analogously to Scheme 2 as described above, although in Scheme 2, the material will shrink and the Young's modulus will increase upon reduction.

Thus, this principle can be used to provide an elongate medical device having controllable stiffness.

According to an embodiment, a first stiffness change component is provided by a change in a moment of inertia of the electroactive polymer material, and a second stiffness change component is provided by a change in the modulus of elasticity of the electroactive polymer material, wherein said first and second stiffness change components counteract each other, and wherein said second stiffness change component is greater than said first stiffness change component.

The modulus of elasticity of the electroactive polymer material may be reducible upon electrochemical reduction, and wherein the stiffness is reducible by applying the electrical signal to induce said reduction of the electroactive polymer material.

Alternatively, the modulus of elasticity of the electroactive polymer material may be reducible upon electrochemical oxidation, and wherein the stiffness is reducible by applying the electrical signal to induce said oxidation of the electroactive polymer material.

The controllable stiffness portion may be formed entirely of the electroactive polymer material.

In one embodiment, the device further comprises a tubular body, wherein the electroactive polymer material is provided on an inwardly and/or outwardly facing surface of said tubular body.

In another embodiment, the device further comprises a tubular body, wherein the electroactive polymer material is provided as in a recess or in a groove of an inwardly and/or outwardly facing surface of said tubular body.

In yet another embodiment, the device further comprises a solid body, wherein the electroactive polymer material is provided on an outwardly facing surface of said solid body.

In yet another embodiment, the device further comprises a solid body, wherein the electroactive polymer material is provided in a recess or in a groove of an outwardly facing surface of said solid body.

In one embodiment, the controllable stiffness portion comprises at least one other material, in addition to the electroactive polymer material.

In one embodiment, the controllable stiffness portion comprises at least two skeletal members, which are substantially aligned in a longitudinal direction of the device.

The device may comprise at least two controllable stiffness portions.

A first one of the controllable stiffness portions may comprise a first type of electroactive polymer material and a second one of the controllable stiffness portions comprises a second, different type of electroactive polymer material.

The controllable stiffness portions may be drivable in opposite phase.

An insulator is arranged between two adjacent controllable stiffness portions.

The electroactive polymer material may extend over a portion of a cross section in a transverse direction of the device, which portion is smaller than the total area of the device cross section. Thus, the electroactive polymer material may be provided within a sector of the device, e.g. with a central angle of less than 180 degrees, preferably less than 90 degrees.

The device may further comprise a at least one further electroactive polymer material extending over a further portion of the cross section in the transverse direction of the device, which further portion is smaller than the total area of the device cross section.

In another embodiment, the electroactive polymer material is provided in a recess in an inwardly and/or outwardly facing surface of the device.

In yet another embodiment, the controllable stiffness portion may be formed entirely of the electroactive polymer material.

The device may be provided with an insulating coating.

In one embodiment, the electroactive polymer material is provided in a controllable device portion that is arranged at a distal end of the device.

In another embodiment the electroactive polymer material is provided in a controllable device portion, and wherein at least one such controllable device portions is interleaved with two non-controllable device portions. A portion that is non-controllable in the sense of this disclosure, may comprise e.g. a tool such as the ones described in WO00/78222.

According to another aspect, there is provided a system comprising an elongate device for introduction into a body lumen as described above, and a control unit, coupled to said electroactive polymer material for providing control signals thereto.

The system may further comprise a counter electrode and an electrolyte, and optionally a reference electrode.

In the system, the counter electrode may be provided on at least one of a controllable portion, a non-controllable portion of the elongate device, and a separate member adapted for introduction into a body lumen.

The electrolyte may at least partially surround the controllable portion.

The electrolyte may be a physiological fluid.

Alternatively, the device may comprise a tubular member, and the electrolyte may be provided inside the tubular member. In one embodiment, the device itself may be a tubular member, whereas, in another embodiment, the device may be provided inside the tubular member.

As yet another alternative, the electrolyte may be provided in the form of a casing or an additional layer on the device.

According to another aspect, there is provided a method for operating an elongate device for introduction into a body lumen, the device comprising an elongate body extending in a longitudinal (L), and having a controllable stiffness portion, comprising an electroactive polymer material, The method comprises controlling the stiffness of the controllable stiffness portion by applying an electrical signal to the electroactive polymer material to change the modulus of elasticity of the electroactive polymer material.

Furthermore, there is provided a method for operating an elongate device for introduction into a body lumen as described above, the method comprising inserting the elongate device into the body lumen and providing electrical signals to the electroactive polymer materials for controlling the shape or stiffness of the elongate device.

A description of embodiments will now be given with reference to the appended drawings.

### BRIEF DESCRIPTION OF THE DRAWINGS

FIGS. 1a and 1b schematically illustrate embodiments of elongate medical devices having controllable portions.

FIGS. 2a-2f schematically illustrate variants of a controllable medical device portion according to a first embodiment.

FIGS. 3a and 3b schematically illustrate a controllable medical device portion according to a second embodiment.

FIGS. 4a and 4b schematically illustrate a controllable medical device portion according to a third embodiment.

FIGS. 5a and 5b schematically illustrate a controllable medical device portion according to a fourth embodiment.

FIGS. 6a and 6b schematically illustrate a controllable medical device portion according to a fifth embodiment.

FIGS. 7a and 7b schematically illustrate a controllable medical device portion according to a sixth embodiment.

FIGS. 8a and 8b schematically illustrate a controllable medical device portion according to a seventh embodiment.

FIGS. 9a and 9b schematically illustrate a controllable medical device portion according to a seventh embodiment.

FIGS. 10a-10b schematically illustrate a controllable medical device portion according to a eight embodiment.

FIGS. 11a-11d schematically illustrate alternative ways of arranging skeletal members in a controllable medical device portion.

FIGS. 12a-12b schematically illustrate a controllable medical device portion according to a ninth embodiment.

FIGS. 13a-13c schematically illustrate a controllable medical device portion according to a tenth embodiment.

FIGS. 13d-13s schematically illustrate further embodiments of a controllable medical device.

FIGS. 14a-14d schematically illustrate alternative elongate medical devices having portions of controllable stiffness.

FIG. 15 schematically illustrates an embodiment of an elongate medical device having a portion of controllable stiffness.

FIG. 16 schematically illustrates another embodiment of an elongate medical device having a portion of controllable stiffness.

FIG. 17 schematically illustrates yet another embodiment of an elongate medical device having a portion of controllable stiffness.

FIG. 18 schematically illustrates yet another embodiment of an elongate medical device having a portion of controllable stiffness.

FIGS. 19a-19b schematically illustrate yet another embodiment of an elongate medical device having a portion of controllable stiffness.

FIGS. 20a-20b schematically illustrate yet another embodiment of an elongate medical device having a portion of directionally controllable stiffness.

FIG. 21 schematically illustrates various placements of a counter electrode relative to an elongate medical device having controllable portions or an elongate medical device having a portion of controllable stiffness.

FIGS. 22a-22c schematically illustrate various electrolyte arrangements for an elongate medical device having controllable portions or an elongate medical device having a portion of controllable stiffness.

FIG. 23 schematically illustrates an insulatingly coated elongate medical device having controllable portions or an elongate medical device having a portion of controllable stiffness.

FIG. 24 schematically illustrates a system according to an aspect of the present disclosure.

FIGS. 25a-25b Schematically illustrates a pair of adjacent skeleton members with an electroactive material arranged therebetween.

### DESCRIPTION OF EMBODIMENTS

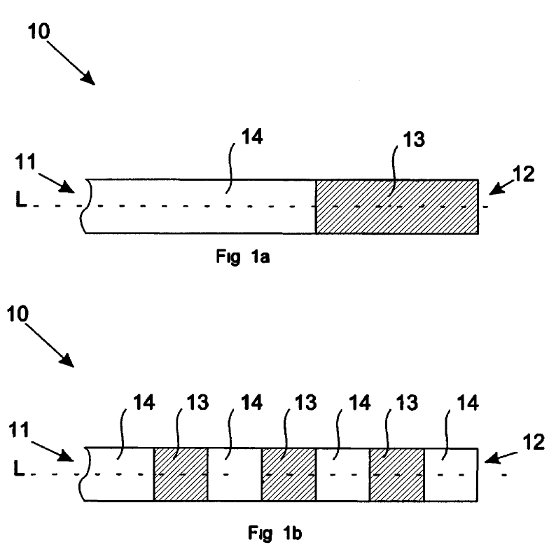

FIG. 1a shows an elongate medical device 10 such as guidewire or catheter, having a proximal part 11 and a distal part 12, which is typically the tip of the device. The distal part 12 comprises an electrically controllable segment 13.

FIG. 1b shows a similar medical device 10, but in this embodiment, the medical device has several electrically controllable segments 13, which are interleaved with non-controllable segments 14.

It is recognized that the embodiments of FIGS. 1a and 1b may be combined, e.g. by providing a medical device having both the interleaved controllable and non controllable segments illustrated in FIG. 1b and the controllable tip illustrated in FIG. 1a.

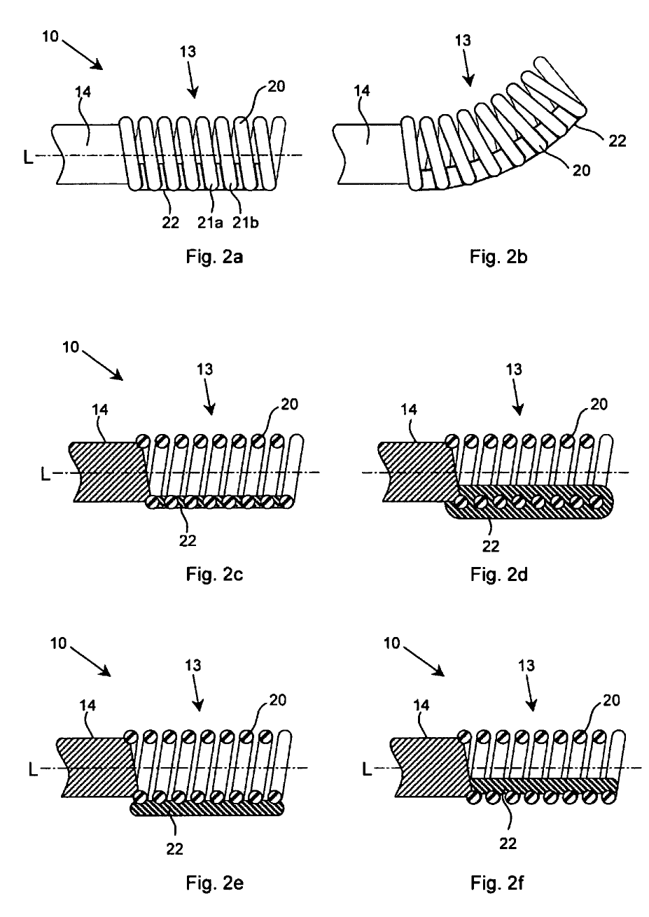

FIGS. 2a and 2b illustrate a first embodiment of an elongate medical device having a non-controllable segment 14 and a controllable segment 13. In this example the segment 13 comprises a helical coil 20, wherein each turn of the coil can be said to form a skeletal member 21a, 21b. The plurality of skeletal members 21a, 21b together form a skeleton or a spine of the device, whereas the EAP material will operate as muscles.

The embodiment is based on the principle of adding bulk EAP material 22 to a portion of the coil 20, linking each turn on the coil together. By having the EAP material distributed asymmetrically or inhomogeneously, a volume expansion of the EAP will result in a bending motion, towards the non-EAP covered side, as is illustrated in FIGS. 2a and 2b.

The EAP material may extend between the two skeletal members 21a, 21b, and contact at least one of the skeletal members 21a, 21b, or, in one embodiment, both. It is contemplated that at least one of the skeletal members 21a, 21b, optionally both, may be provided with a coating, such that the EAP material contacts the coating instead of, or in addition to, contacting the member 21a, 21b.

In one embodiment, the device comprises at least tree, preferably more, longitudinally spaced apart skeletal members 21a, 21b, and the electroactive polymer material extends over these at least tree, or more, skeletal members 21a, 21b.

In FIGS. 2a and 2b, the EAP material extends over a portion P1, P2 (FIGS. 25a-25b) of a cross section of the skeletal members 21a, 21b, the cross section forming a plane perpendicular to the longitudinal direction L of the device 10, termed “transverse cross section”, which portion P1, P2 is smaller than the total area of the transverse cross section of the skeletal members 21a, 21b. For example, the portion P1, P2 may be less than 95% of the total area of the transverse cross section of the skeletal members 21a, 21b, less than 90%, less than 80%, less than 75%, less than 60%, less than 50%, less than 35%, less than 25%, less than 20%, less than 15%, less than 10%, or less than 5%.

The EAP material may be arranged as an elongate body of material, extending over several of the longitudinally spaced apart skeletal members 21a, 21b. In the relaxed state of the EAP, the device may be as shown in FIG. 2a, i.e. substantially straight, along line L, or bent in an opposite direction to that of the activated device. When the EAP is activated, i.e. reduced or oxidized, the device transforms into the state shown in FIG. 2b, wherein the device is bent in a plane containing the EAP material. In the example shown in FIGS. 2a and 2b, an expanding EAP material is used.

The amount of bending may be controlled by selecting an appropriately doped EAP, by selecting the number of skeletal members 21a, 21b along which the EAP material extends, by selecting the type of coil and coil properties, by selecting the gap between the skeletal members 21a, 21b and by controlling the extent to which the EAP is reduced or oxidated.

As an example, the EAP material could be a conducting polymer such as PPy(DBS). In order to activate, i.e. bend the tip, a negative potential of about 0 to −5 V, typically about −1 V, can be applied to the PPy. The EAP material thereby reduces and swells by taking in cations such as Na+, according to scheme 1 as discussed above. Hence, the electroactive polymer material may be expandable when subjected to an externally applied electrical signal. Applying a zero or slightly positive (0 to +5, typically +0.5 V) potential, the PPy shrinks and the controllable section 13 straightens again. This process can be repeated many times. Other non-limiting examples of EAP material are electrically activated hydrogels (T. Hirai, J. Zheng, and M. Watanabe, “Solvent-drag bending motion of polymer gel induced by an electric field”, in Smart Structures and Materials, EAPAD '99, 1999, Newport Beach, Calif., USA, Proceedings of SPIE, p. 209-217; and P. Calvert and Z. Liu, “Electrically stimulated bilayer hydrogels as muscles”, in Smart Structures and Materials, EAPAD '99, 1999, Newport Beach, Calif., USA, Proceedings of SPIE, p. 236-241.) or carbon nanotubes (G. M. Spinks, et al., “Pneumatic carbon nanotube actuators”, Advanced Materials, 2002, 14(23), p. 1728).

In FIG. 2c, a variant of the first embodiment is shown, wherein the EAP material is present only in the space between portions P1, P2 (FIGS. 25a-25b) of the skeletal members 21a, 21b, as opposed to the variant of FIG. 2d, wherein the EAP material is arranged between and surrounding the portions of the skeletal members 21a, 21b.

Thus, in FIG. 2c, the skeletal members are formed from the turns of a coil, whereby the EAP material is present in a spaced defined by two longitudinally aligned and spaced apart portions of two turns of the coil. The EAP material of FIG. 2c may be present only in this space and possibly only in part of this space.

In FIG. 2d, the skeletal members are formed from the turns of a coil, whereby the EAP material is present in the spaced defined by two longitudinally aligned and spaced apart portions of two turns of the coil. The EAP material of FIG. 2d may enclose the portions of the turns of the coils and forms a continuous body extending over two or more turns of the coil.

In FIG. 2e, the EAP material 22 is provided substantially on an outside of the coil 20, whereas in FIG. 2f, the EAP material 22 is provided substantially on an inside of the coil.

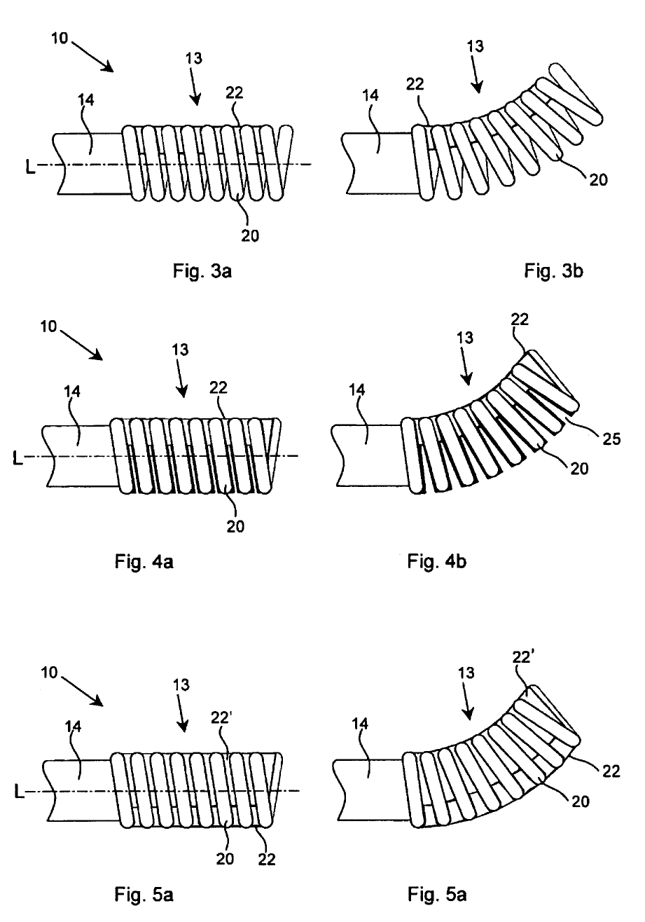

FIGS. 3a and 3b show another embodiment. Similar to the previous embodiment (FIGS. 2a and 2b), only a part of the coil's transverse cross section has been covered with EAP material. In this embodiment, the EAP material 22 contracts on electrical stimulation resulting in a bending towards the EAP side. Hence, the EAP material is contractable when subjected to an externally applied electrical signal. An example of such an EAP is PPy doped with a small, mobile anion, as is described in scheme 2.

The PPy(DBS) can be applied to the coil in several ways. For instance, the complete coil can be covered with PPy(DBS), e.g. by using electrochemical synthesis as is known to those skilled in the art, following which a part of the PPy is removed to provide the embodiment disclosed in FIGS. 4a and 4b.

Alternatively, a part of the coil can be covered by an insulating material leaving only the part of the coil where the PPy should be synthesised exposed to the synthesis solution, whereby PPy is only added on that part, resulting in the embodiments of e.g. FIGS. 2a-2b, 3a-3b. The insulating material may be left on the coil, or it may be selectively removed, such that all parts of the coil that are not covered by the EAP material are exposed.

FIGS. 4a-4b illustrate the embodiment where EAP material has been provided on the entire coil and thereafter removed. An EAP body 22 has initially been applied to the entire coil 20. A portion of the EAP 22 between two adjacent skeletal members 21a, 21b has then been removed to create an unsymmetrical EAP distribution, where the lower parts of the skeletal members 21a, 21b (as seen in the figure) of the coil are not linked by the EAP-material, and wherein a gap 25 is present. As the EAP 22 contracts or expands, the coil will bend. In this example the EAP could be PPy doped with a small anion as explained in scheme 2, but it may also use the principle of scheme 1. Such removal is possible by e.g. laser ablation or reactive ion etching.

Yet another way of creating the asymmetric volume expansion of EAP material 22 is by destroying, degrading or passivating parts 22′ of the EAP material, thereby making it less active or inactive. Such passivation is known by those skilled in the art. FIGS. 5a and 5b schematically show this embodiment with an expanding EAP as an example. The EAP material 22 is dispersed along the coil 20. Parts 22′ of the EAP material have been rendered less active or inactive. So when a potential is applied, only the unaffected part 22 of the EAP material expands and the device bends towards to degraded part 22′.

Alternatively to passivating parts of the EAP material that are not to change their volume, it is possible to instead, or as a compliment, improve those parts of the EAP material that are to change their volume.

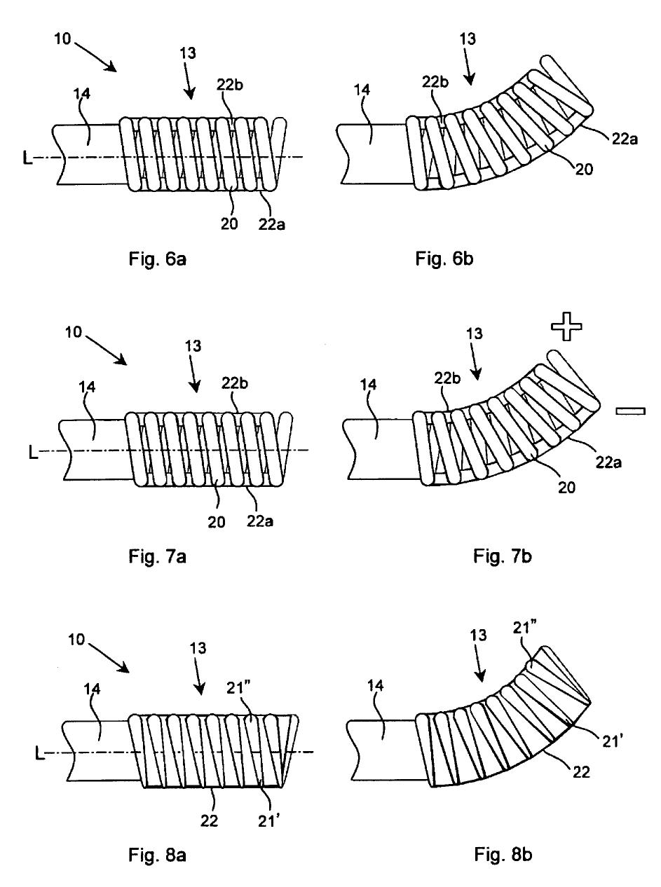

In another embodiment, the “coil” is provided on two sides with two different types of EAP 22a and 22b, e.g. diametrically opposed to one another as shown in FIGS. 6a and 6b. The two different types of EAP have opposite behavior, one 22b contracts on electrical activation and the other 22a expands thus collaborating and generating a bending of the segment (13). This could be achieved for instance by choosing a version of PPy according to scheme 1 for the expanding part 22a and a version of PPy according to scheme 2 for the contracting part 22b.

In an embodiment similar to the previous embodiment, and which is shown in FIGS. 7a and 7b, the EAP material is the same on both sides but is driven in opposite phase, in what is called the “rocking chair configuration”. The two EAP materials 22a, 22b are electrically insulated from one another. Opposite potentials are applied to the EAP materials 22a, 22b. Taking PPy(DBS) (scheme 1) as an example, one side 22b is oxidized by applying a positive potential resulting the shrunken (contracted) phase of PPy(DBS), while the other side 22a is reduced, giving the expanded phase of PPy(DBS), thus resulting in a bending motion towards the shrunken PPy side 22b. In this case, the need for a counter electrode can be eliminated, since the EAP portions 22a, 22b may act as working electrode and counter electrode, respectively.

FIGS. 8a and 8b illustrate an embodiment of the invention, where the coil is not symmetrical. In the example of FIG. 8a, the lower part 21′ of the skeletal member is thinner than the upper part 21″ of the respective skeletal member, making it easier to bend. Such a coil may be formed by using a thread having a varying cross section, or by removing parts of the coil 20 material subsequent to having formed the coil 20. Thus, the cross section of the skeletal members 21a, 21b vary over its rotation about the longitudinal axis of the coil. EAP material 22 may be provided on the entire coil, and as the EAP material 22 changes volume, it is mainly the lower side of the coil 20 that moves, thereby creating the bending motion. This is partly because that side of the coil is weaker, partly because there is a larger volume of EAP material on that side, which is changing volume.

In FIGS. 9a and 9b, a coil formed from two different materials having different elasticity properties is provided. Each turn of the coil presents a portion 21d having higher stiffness and a portion 21c having lower stiffness. An EAP body 22 has been applied to the entire coil 20. When the EAP material is activated, the coil will bend to one side, since the bending stiffness of different portions of each coil turn varies. Thus, bending will occur towards the stiffer side. In this embodiment, the EAP material may be applied as is described with reference to any one of FIGS. 2a-9b.

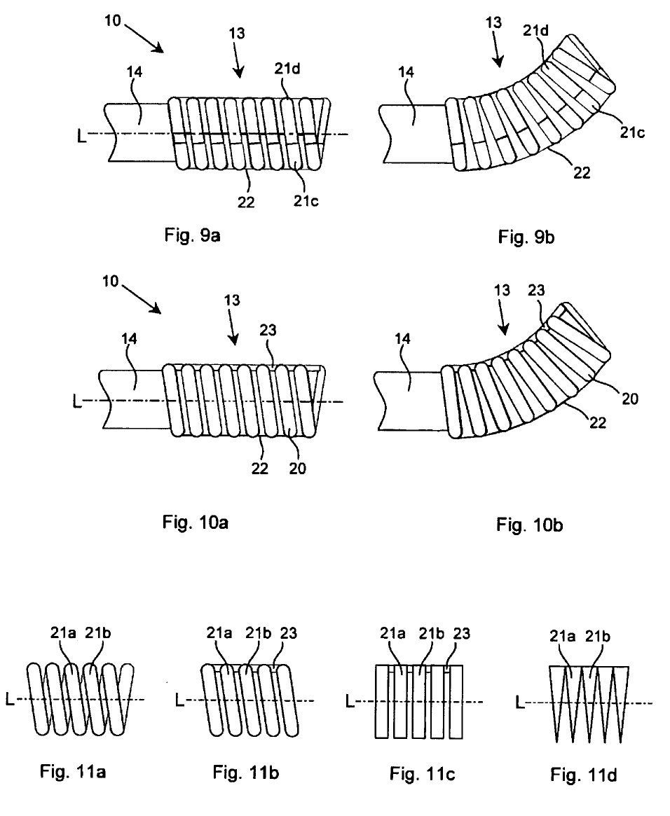

FIGS. 10a-10b show another example of a coil having asymmetric properties. In this case, the coil loops are linked together along an axis parallel with the longitudinal axis of the coil, creating asymmetrical coil stiffness. The linking can be provided by a longitudinal link member 23, such as a wire or rod 23 any suitable material, which is arranged to link coil segments that are longitudinally spaced apart but substantially adjacent. As the EAP material 22 expands, the device bends towards the linked side. The wire or rod 23 may be of a material other than an electroactive polymer material, and it may form an axis substantially parallel with the longitudinal direction of the device.

Referring to FIGS. 11a-11d, descriptions of alternative embodiments of the coil 20 will now be given.

FIG. 11a illustrates a common type of symmetric coil, formed from a wire having a circular cross section. It is recognized that the wire may have any suitable cross section, e.g. elliptic, square, regtangular etc. Two longitudinally spaced-apart portions of the skeletal members 21a, 21b, or coil segments, are longitudinally aligned.

FIG. 11b illustrates an alternative embodiment, wherein a plurality of discrete annular members 21a, 21b are placed in a mutually longitudinally spaced relationship. The members 21a, 21b may be placed at an oblique angle relative to the longitudinal axis L of the device. The members 21a, 21b may have any suitable form, e.g. annular, torus, toroid, quoit, or disk-shaped. The members may optionally be linked to each other along an axis parallel with the longitudinal axis of the device by a longitudinal link member 23, thereby providing asymmetric stiffness properties.

FIG. 11c illustrates another alternative embodiment, wherein a plurality of discrete annular members 21a, 21b are placed in a mutually longitudinally spaced relationship. The members are placed at an angle that is perpendicular to the longitudinal axis L of the device. The members may optionally be linked to each other along an axis parallel with the longitudinal axis of the device by a longitudinal link member 23, thereby providing asymmetric stiffness properties.

FIG. 11d illustrates yet another alternative embodiment, wherein a plurality of discrete members 21a, 21b are placed in a mutually longitudinally spaced relationship. The members 21a, 21b have a thickness that varies as seen in a direction perpendicular to the longitudinal axis L of the device, thereby providing asymmetric stiffness properties. The members 21a, 21b may be linked to each other as is indicated in the upper part of FIG. 10d.

The embodiments of FIGS. 11b-11d may be produced by assembling a plurality of discrete members and, optionally, linking them by providing link members 24.

As another option, the embodiments of FIGS. 11b-11d may be produced by using a tubular rod as starting material, wherein appropriate portions of the rod are removed to form the structure indicated in FIGS. 11b-11d.

The members shown with respect to FIGS. 11a-11d may have any cross section, e.g. circular, elliptic, square, rectangular, triangular, cruciform, semi-circular or semi-elliptic, etc. The members may be hollow or solid. The members in the embodiment of FIG. 11b or 11c may be longitudinally linked or non-linked.

The members 21a, 21b and/or the coil 20 may be formed from any material, such as metal, polymer, rubber or combinations thereof etc., having the required stiffness properties.

The EAP material may be added after the members 21a, 21b and/or the coil 20 have been formed.

In the configuration indicated in FIG. 1b, different controllable segments 13 may be formed according to different principles, selected from those described herein.

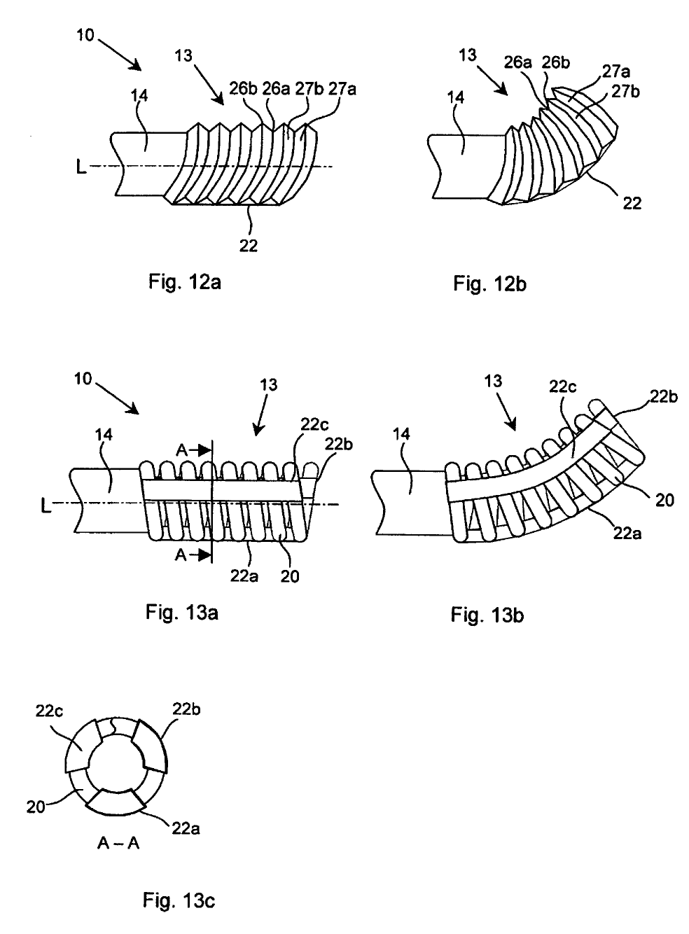

FIGS. 12a and 12b illustrate yet another embodiment, wherein the skeletal members are separated by inner and outer circumferential creases 26a, 26b, and whereby the skeletal members 27a, 27b are flexible enough to enable the device to bend, in a manner resembling a vacuum cleaner hose or an accordion. A portion of the inside and/or outside surface of the device is provided with the EAP material 22. The portion of the surface may have an axial extension along the longitudinal axis of the device

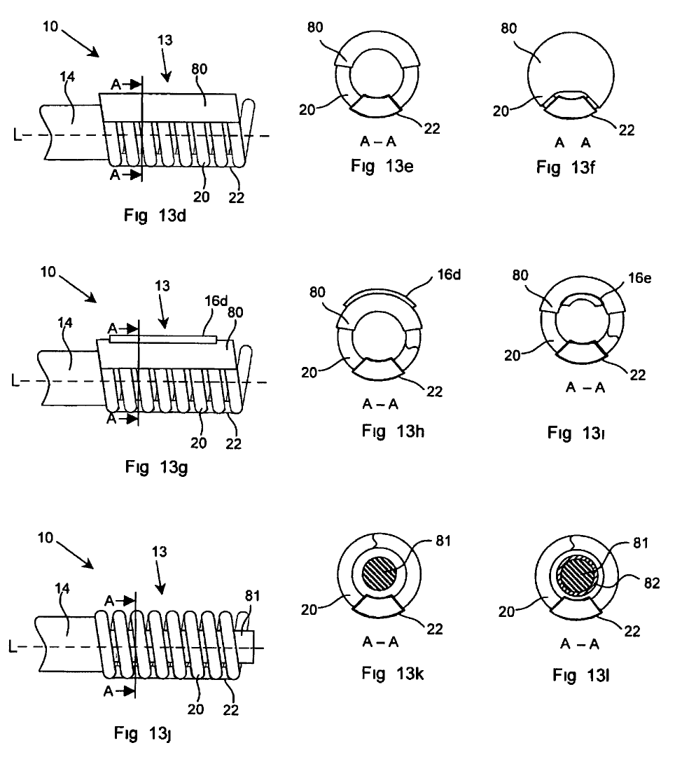

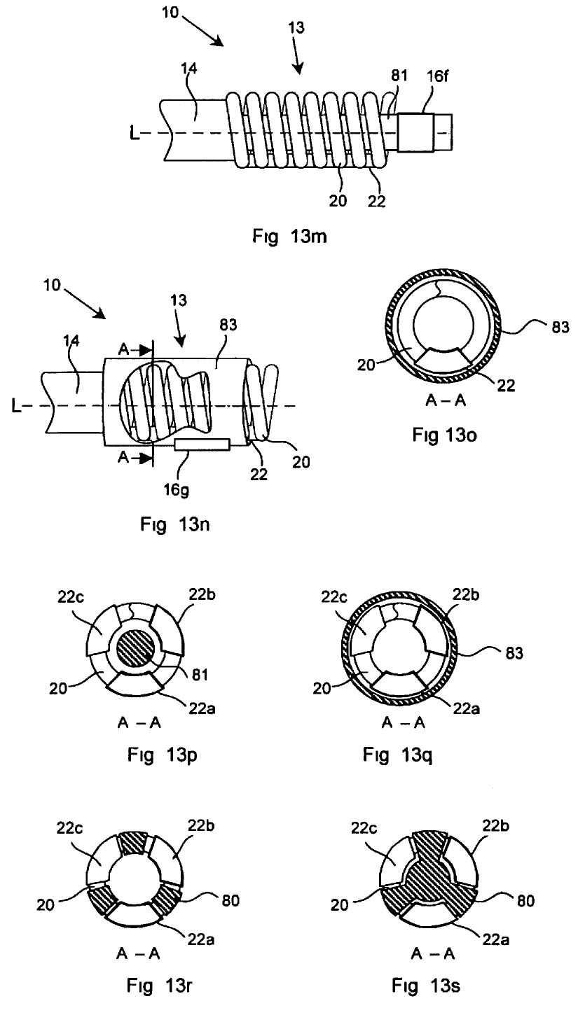

The previously shown examples illustrates motion in one direction perpendicular to the longitudinal axis L of the device. Motion in two directions perpendicular to the longitudinal axis L of the device can be achieved by providing multiple EAP sections or by dividing the EAP section into three, four or more sections as is illustrated in FIG. 13a-13c. Each section 22a, 22b, 22c contains electroactive polymer that is individually controllable.

The sections 22a, 22b, 22c may be electrically insulated from one another (not shown in the drawing). In the example with three controllable sections 22a, 22b, 22c each EAP section 22a, 22b, 22c is driven individually. In the four segments example (not shown), each EAP section may be driven individually, or opposing EAP sections may be driven antagonistically, e.g. as illustrated in FIGS. 6a-6b or 7a-7b.

FIGS. 13d-13e illustrate another embodiment of the present disclosure, wherein a controllable portion 13 comprises a coil 20 provided with an EAP material 22 at a portion thereof, as seen in a cross section A-A perpendicular to a longitudinal axis L.

As mentioned before, a part of the coil can be covered by a covering material 80 leaving only the part of the coil where the EAP material should be synthesized exposed to the synthesis solution, whereby EAP material is only added on that part. The covering material 80 may be insulating, e.g. a polyurethane, a silicone, epoxy, or it may be ion conducting material, such as NAFION®, FLEMION® etc, or combinations thereof.

Leaving this covering material 80 on the coil 20 after the EAP synthesis has several advantages. First, it makes the fabrication less complex as one process step (removing the covering) is eliminated. Second, it may improve the durability of the device since it reduces the risk of kinks in the coil. Third, if the covering material 80 is made from an electrically insulating material, then it may also be used as a base for providing wiring and/or electrodes. Hence, it may allow the CE 16 to be integrated on the coil 20.

FIG. 13e, which is a cross section along line A-A in FIG. 13d, illustrates a covering material 80 that only covers a part of the wire forming the coil, whereas FIG. 13f, which is a modified cross section A′-A′ at line A-A in FIG. 13d, illustrates a coil which is entirely covered and filled with material 80, except for the part covered by the EAP material.

The covering material 80 may cover part of the coil, as illustrated in FIGS. 13d-13e, or, in a non-illustrated embodiment, the entire coil, except for the part which is covered by the EAP material, while the coil still presents an axial channel, similar to the one illustrated in FIG. 13e. The material may also wholly or partially cover the EAP material.

FIGS. 13g through 13i illustrated how a CE 16 may be arranged on the coil 20 by placing the CE on an insulating covering 80. In FIGS. 13g and 13h (cross section) the CE 16d is placed on the outside and in FIG. 13i, which is a modified cross section A′-A′ at line A-A in FIG. 13g, the CE 16e is placed on the insulating covering 80, but on the inside of the coil 20.

Yet another approach to increasing the durability of the bending medical device is to add a rod-like structure substantially concentrically inside the coil 20. This is schematically illustrated in FIGS. 13j through 13m.

The rod 81 may be of any shape or material as long as it reduces kinking. It may be a solid material, a tubular structure, a wire etc. It may even be made from a porous material or an ion conducting material, such as NAFION®, FLEMION® etc, or combinations thereof.

The rod 81 may be made from a conducting material, such as a metal, or an insulating material, such as a polymer. Making the rod 81 non-conducting gives the opportunity to further integrate other parts of the electrochemical system such as a counter electrode 16f as illustrated in FIG. 13m and/or a reference electrode (not shown).

In the embodiment disclosed in FIG. 13j, and 13k, a solid rod 81 is positioned inside the coil 20 provided with an EAP material 22.

In the embodiment disclosed in FIG. 13l, which is a modified cross section A′-A′ at line A-A in FIG. 13j, the rod is conducting and provided with an electrically insulating but ionically conducting covering 82, which enables use of the rod 81 itself as counter electrode or reference electrode, while the EAP material 22 forms the working electrode. Such electrically insulating but ionically conducting coverings may be provided by materials, such as NAFION®, FLEMION® etc, or combinations thereof, or in the form of an insulating mesh, grid, spacers or porous structure. Examples of such materials comprise porous materials such as Keralpor 99® from KERAFOL®-Keramische Folien GmbH, Eschenbach i.d. Opf, Germany. Teflon filters, teflon mesh, etc may also be used. As another option, it may be provided in the form of an insulating structure that is patterned so that nanometer or micrometer wide channels are created that can conduct the electrolyte from the space wherein it is housed to the electrode. This insulating, patternable layer may be fabricated using materials such as SU8, BCB (benzocyclobutene Cyclotone®), or polyimide, either by direct photopatterning or by removing material by etching.

The substantially concentric rod may also reduce the risk for kinks in the coil. The rod may be combined with the insulating covering 80 disclosed in FIGS. 13d-13e.

Alternatively, or as a complement to the covering material 80 and the rod 81, as illustrated in FIGS. 13n-13o, a tubular structure 83 or casing may be provided around the coil 20, being substantially concentric with the coil 20.

FIG. 13n illustrates a such a casing, which is partially broken away for illustration purposes. The tubular structure may, in one embodiment, be porous, a net, or a mesh structure, in order to permit ion and solvent contact with the electrolyte surrounding the device. In the embodiment of FIG. 13n, an electrode 16g may be arranged on the casing 83.

In another embodiment, the tubular structure 83 may be closed so that electrolyte may be contained within the tubular structure 83. Hence, the bending tip, including the CE and possibly the RE, may provide an encapsulated system. An electrode may be provided on the casing 83.

FIGS. 13p-13s, which are modified cross sections A′-A′ at line A-A in FIG. 13a, illustrate embodiments, wherein a plurality of EAP material sections 22a, 22b, 22c are provided in a manner similar to the embodiments disclosed in FIGS. 13a-13c.

In the embodiment of FIG. 13p, a rod 81 is provided inside the coil 20 in order to reduce the risk of kinking.

In the embodiment of FIG. 13q, a tubular structure 83 is provided around the coil 20, in order to reduce the risk of kinking, and/or in order to provide an encapsulated system.

In the embodiment of FIG. 13r, the coil 20 is covered by insulating material portions 80, analogously with the embodiment described with respect to FIGS. 13d-13e.

In the embodiment of FIG. 13s, the coil is covered and filled by insulating material 80, analogously with the embodiment described with respect to FIG. 13f.

The description will now be directed to the second aspect, i.e. to providing an elongate medical device having controllable stiffness. Such devices include, but are not limited to, catheters and endoscopes.

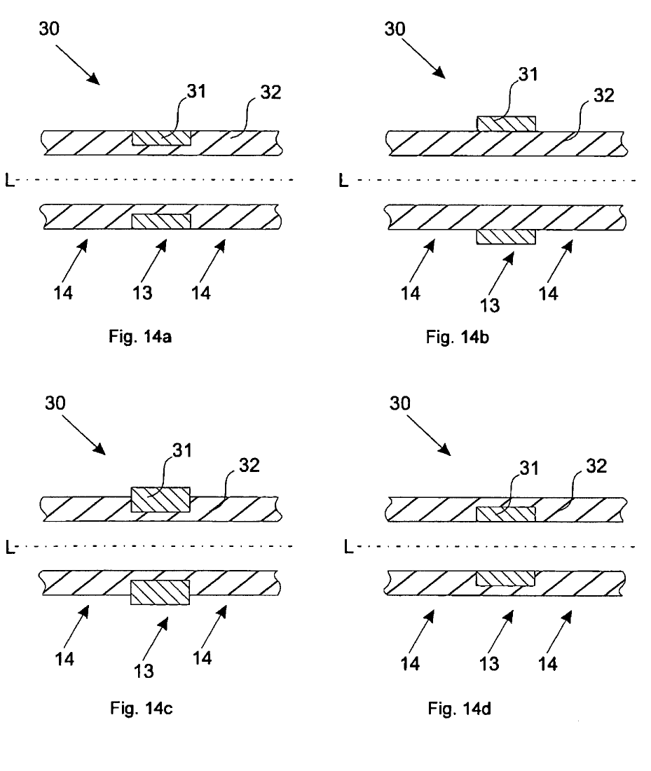

FIGS. 14a-14d illustrate cross sections of embodiments of a tubular device 30 having an elongate hollow body 32, whose stiffness is substantially fixed at any given temperature, and having a body portion 13, comprising an EAP material 31. By controlling the redox state of the body portion 13, the moment of inertia of the body portion 13 is controlled, whereby the device can be rendered more or less stiff. The EAP material can be added to the device or replace parts of the device.

In FIG. 14a, an annular groove around the periphery of the device has been provided at a portion of the elongate body 32, thereby providing a portion of the elongate body having a decreased wall thickness. In this annular groove, an electroactive polymer material 31, such as polypyrrole, has been arranged. Upon activation (e.g. electrochemical reduction) of the EAP material, the Young's modulus reduces and the segment becomes less stiff, allowing for a better bending in curvatures of e.g. arteries etc. The reduction of PPy is accompanied by an increase of the volume of the PPy ring. However, if the device is designed such that the product of E*D̂4 is less in the reduced state, the reduction in Young's modulus will dominate. If the polypyrrole is oxidized, the Young's modulus will increase, making the device more stiff resulting in a good pushability, e.g. in connection with penetration of obstructions.

FIG. 14b illustrates a second embodiment, wherein the EAP material 31 has been arranged to form an annular protrusion around the periphery of a portion of the elongate body 32. In this embodiment, the EAP material will, in its passive state, act so as to reinforce and increase the stiffness of the elongate device, whereby this increase in stiffness is reduced upon activation (reduction or oxidation) of the EAP material.

Table 1 below illustrates an example based on the embodiment disclosed in FIG. 14b, wherein catheter diameters ranging from 1.98-2.67 mm in diameter have been provided with a PPy layer of 0.05 or 0.1 mm thickness. The diameter in meters of the catheter before and after oxidation and the stiffness of the catheter in Pa*m⁴ is set forth in the respective case. The stiffness S has been defined as the product of the moment of inertia I and the modulus of elasticity E, i.e. the Young's modulus, S=IE. The stiffness of the EAP layer was calculated assuming that the layer has a tubular form, with the inner diameter Dᵢₙ being identical to the outer diameter of the respective catheter and the outer diameter Dₒᵤₜ, in the neutral case, being defined as Dₒᵤₜ=Dᵢₙ+2T_{EAP}, where T_{EAP} is the thickness of the EAP layer. Furthermore, it was assumed that the volume increase of the EAP layer when activated is 20%. Hence,

$${S}_{EAP} = \frac{π}{64} E ( {D}_{out}^{4} - {D}_{I n}^{4} ) .$$

Furthermore Eₒₓ was assumed to be 200 MPa and E_{red} was assumed to be 500 MPa.

The calculations have been made for annular members on catheters having dimensions French 6 (1.98 mm diameter), French 7 (2.31 mm diameter) and French 8 (2.67 mm diameter).

TABLE 1
diameter and stiffness of PPy layer of FIG. 14b

| Catheter dimensions | | | | | | | | |
|---|---|---|---|---|---|---|---|---|
| | | Ppy layer | Oxidated state | | Reduced state | | | |
| | Diameter | thickness | Diameter | Stiffness | Diameter | Stiffness | Diam | Stiff |
| French | [m] | [m] | [m] | [Pa*m\hat{ }4] | [m] | [Pa*m\hat{ }4] | change | change |
| 6 | 1.98E−03 | 5.00E−05 | 2.08E−03 | 8.21E−05 | 2.10E−03 | 4.00E−05 | 1.00% | 48.70% |
| 7 | 2.31E−03 | 5.00E−05 | 2.41E−03 | 1.29E−04 | 2.43E−03 | 6.27E−05 | 0.80% | 48.60% |
| 8 | 2.67E−03 | 5.00E−05 | 2.77E−03 | 1.98E−04 | 2.79E−03 | 9.59E−05 | 0.70% | 48.50% |
| 6 | 1.98E−03 | 1.00E−04 | 2.18E−03 | 1.77E−04 | 2.22E−03 | 8.75E−05 | 1.80% | 49.40% |
| 7 | 2.31E−03 | 1.00E−04 | 2.51E−03 | 2.75E−04 | 2.55E−03 | 1.35E−04 | 1.60% | 49.20% |
| 8 | 2.67E−03 | 1.00E−04 | 2.87E−03 | 4.18E−04 | 2.91E−03 | 2.05E−04 | 1.40% | 49.10% |

As appears from Table 1, the increase in diameter of the PPy ring of FIG. 14b is in the magnitude of 1%, whereas the decrease in stiffness is in the magnitude of 50%. Similar behavior is expected from the embodiments described and discussed with reference to FIGS. 14a-20b.

FIG. 14c illustrates a combination of FIGS. 14a and 14b, wherein the EAP material is provided in a groove, just like in FIG. 14a, but wherein the EAP material 31 also protrudes annularly from the periphery of the elongate body 32.

FIG. 14d illustrates an embodiment, wherein the EAP material is provided in an annular groove on the inner surface of an elongate tubular device 30. This embodiment will operate similar to that of FIG. 14a, but as the EAP material is activated, it will also decrease the inner cross section of the device 30.

The EAP material may be provided in an annular shape as is shown in the previous figures, but also in a spiral shape, or any other shape or section. In particular, one or more EAP material portions may be provided, e.g. such as is shown in FIGS. 1a or 1b. The EAP parts do not need to have a uniform thickness. They can be tapered or have any type of variable thickness, e.g. varying in a stepwise manner.

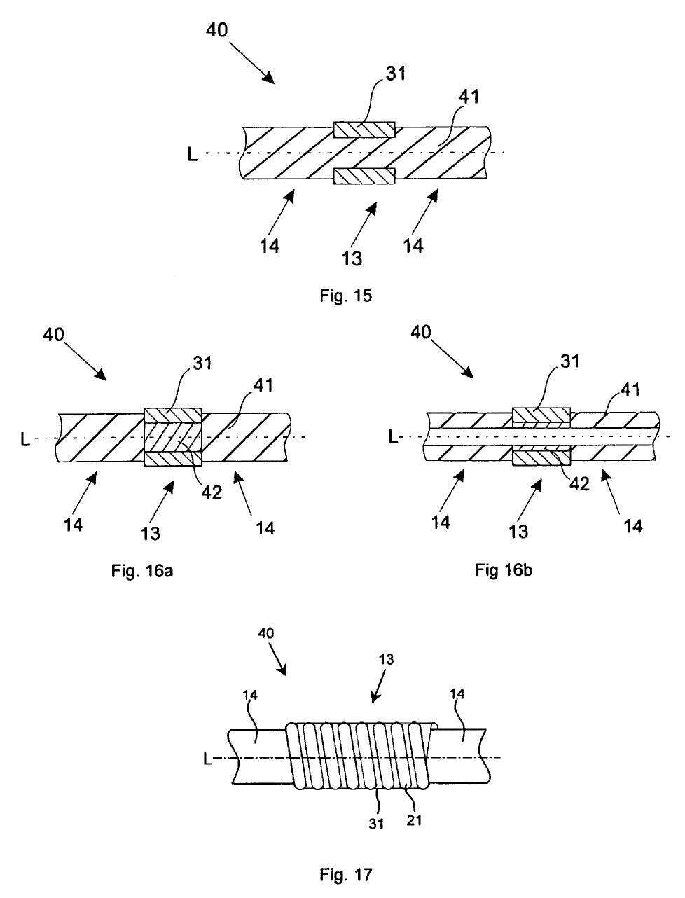

The invention is not limited to tubular devices. FIG. 15 shows an example of a solid rod or wire, such as a part of a guidewire, where EAP material 31 has been added to a segment of the wire 40 in a groove similar to that of FIG. 14a or 14c. Other device shapes are contemplated as well as other placements, e.g. as is shown in FIGS. 14a-14d.

The elongate device does not have to consist of a single material. FIG. 16a illustrates an example of a device 40 that contains multiple materials 31, 41, 42. In this example, the elongate device not only comprises the EAP material 31 and the elongate body 41, but a third material 42 is present, in the segment 13 of the elongate device 40 having an electrically controllable stiffness. Multiple materials are contemplated as well.

FIG. 16b illustrates an example of an embodiment similar to that of FIG. 16a, but where the device 40 is tubular, and so the controllable portion 13 comprises two substantially concentric annular material portions, one of which being the EAP material 31.

In yet another embodiment, which is illustrated in FIG. 17, an existing open space in the controllable segment 13 of the device is filled with the EAP. The device may be similar to that of FIG. 2a-2b, in that a coil structure 20, for instance at a catheter tip, is covered with an electroactive polymer 31, so that the “empty space” between the turns of the coil is filled with the material, thereby interconnecting the separate elements. Reducing or oxidizing the material changes the overall bending stiffness of the material and thus has an impact on coil flexibility or pushability. This embodiment may be provided as a tubular (hollow) device or as a solid device, depending on how the EAP material is arranged.

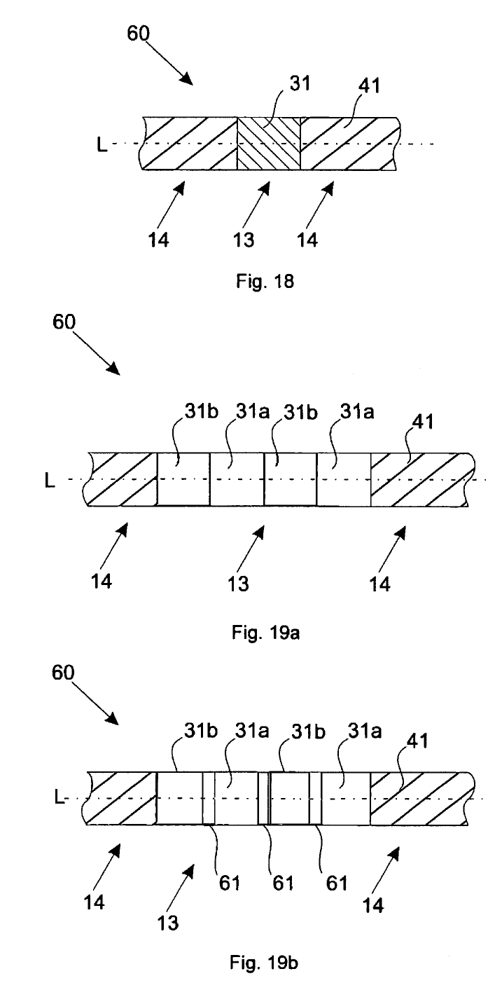

In yet another embodiment, the entire controllable segment 13 is made of Young's modulus changing material, such as EAP material. FIG. 18 shows an example of this embodiment. In this example the device 60 is a rod or wire, but the device 60 can just as well have other shapes, such as a tube or a catheter. The controllable segment 13 may thus be completely made of EAP material 31.

FIGS. 19a-19b illustrate an embodiment of an elongate device 60 having a controllable segment 13 comprising several parts 31a, 31b that change from a neutral state to an altered Young's modulus, similar to FIG. 18.

In FIG. 19a, the EAP segments 31a, 31b of the device 60 comprise two different EAP materials 31a and 31b. As a non-limiting example, a first EAP material 31a could be PPy according to scheme 1, such as PPy(DBS) and a second EAP material 31b could be PPy according to scheme 2, such as PPy(ClO4). Starting from a position wherein both materials are in a neutral state, applying the same electrical stimulus (redox potential) to both parts 31a, 31b, the first parts 31a become softer and the second parts 31b become more rigid (or vice versa) than in the neutral state, thus making the whole device more flexible.

In FIG. 19b, the elongate device 60 contains the first and second EAP material parts 31a and 31b that are made from the same material, such as PPy(DBS). They are electrically insulated from one another by an insulating member 61, and the first and second parts 31a, 31b can be driven in opposite phase by applying opposite potentials, according to the so called “rocking chair mode”. From a neutral state, the first part 31a becomes oxidized, and thereby more stiff and the second part 31b becomes reduced, and thereby more flexible. The whole segment thus becomes more flexible. In one embodiment of such a system, the material of the first and second parts 31a, 31b is selected so that they have approximately the same stiffness in the neutral state.

The previous embodiments show a stiffness change along the device circumference, having no predetermined or preferred direction of stiffness change. It might be advantageous to be able to direct the change of stiffness to a certain direction of the elongate medical device.

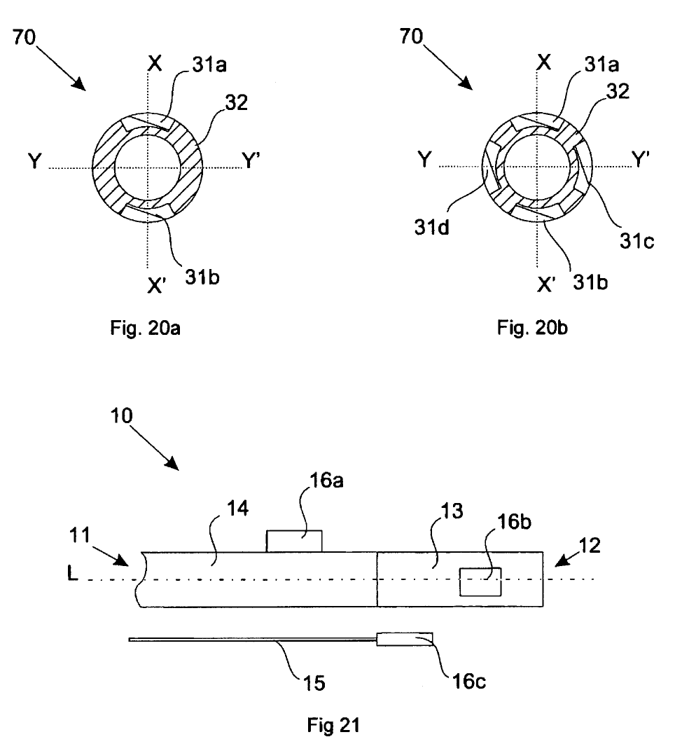

FIG. 20a-20b illustrate a cross section of an elongate tubular device 70, where only a part of the circumference is provided with the EAP material having a controllable elastic modulus.

In the embodiment illustrated in FIG. 20a, the EAP material is provided as longitudinal strips of EAP material, arranged in longitudinal grooves in the walls of the device 70. Alternatively, the strips may be provided on the periphery of the device 70, analogously to what is shown in FIG. 14b. The stiffness change is only affected in one dimension, i.e. in a direction perpendicular to the EAP parts 31a, 31b, in the direction X-X′. The stiffness in the direction Y-Y′ remains unchanged. If the parts 31a, 31b become softer on electrical stimulus, such as PPy(DBS), scheme 1, bending in the direction X-X′ is favored. If, on the other hand, these parts 31a, 31b, become harder, scheme 2, bending in the direction Y-Y′ is favored.

In the embodiment illustrated in FIG. 20b, the bending stiffness can be altered in two directions, since two pairs 31a, 31b; 31c, 31d of mutually oppositely arranged strips are provided, in a manner similar to that described with reference to FIG. 20a. For example, a pair of strips 31a, 31b, 31c, 31d may be mutually diametrically oppositely arranged.

It is contemplated that devices having a configuration similar to that of FIGS. 20a-20b but with three, four, five or more EAP material parts can be provided.

Different drive schemes are possible in respect of the embodiments shown in FIGS. 20a and 20b. If all EAP material parts 31a, 31b, 31c, 31d of a controllable stiffness segment are in the relatively stiff phase, then good pushability is achieved. If only one part, 31a or 31b or 31c or 31d, is in the soft phase and the others are in the stiff phase, then flexibility to one direction is achieved (for example, bending towards or from the soft part side). If all parts are in the softer phase, then good flexibility in all directions is achieved, for instance to follow curves.

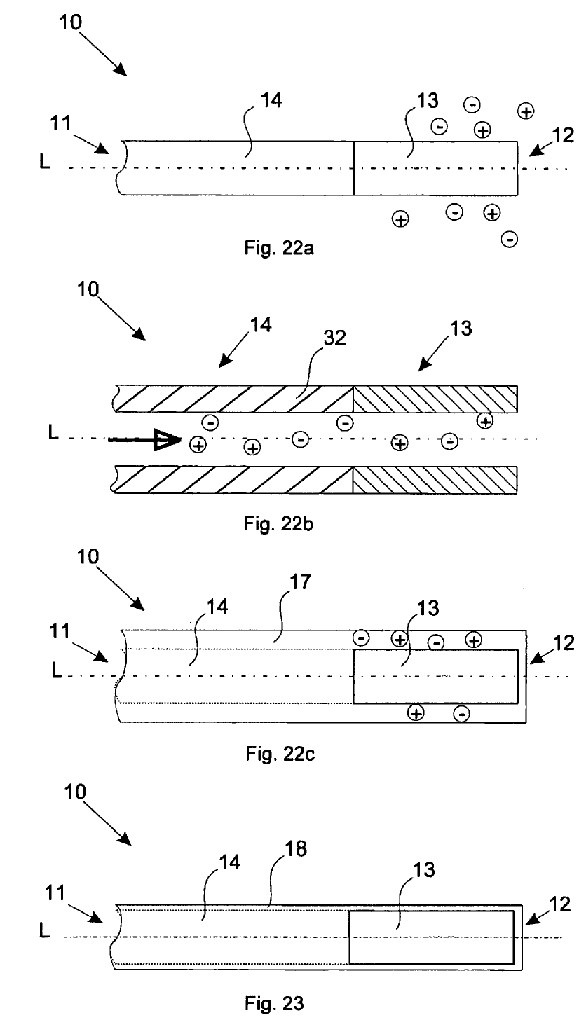

The devices described in this disclosure could be of a non-conducting material, such as a plastic. In those cases, the conducting polymer could be adhered to a conducting substrate, such as gold or to another conducting polymer. This substrate has been omitted from the figures to increase clarity of these principle sketches. Also, electrical leads (not shown in the sketches) may be included in the devices in order to address and electrically contact the conducting polymers with the power source and control unit. A counter electrode 16a, 16b, 16c and possibly even a reference electrode (not shown) may be included in, on, or near the devices for a complete control. These electrodes can be integrated on the device or provided separately, as is schematically illustrated in FIG. 21.

FIG. 21 schematically illustrates possible examples of where the counter electrode 16a, 16b, 16c could be placed in or on the medical device 10 having a controllable segment 13.

Using a separate device 15 such as a second catheter, a guidewire, a lead etc., the counter electrode 16c may be positioned near the first medical device 10.

Alternatively, or as a complement, the counter electrode 16a may be placed on a non-controllable portion 14 of the device 10.

Alternatively, or as a complement, the counter electrode 16b may be placed on an electrically insulated part of the controllable portion 13 of the device 10.

Furthermore, for the EAP to be operable, an electrolyte is needed. The electrolyte functions as an ion source/sink and establishes a closed conducting path for the electrical current from the working electrode to the counter electrode.

The electrolyte could be a physiological fluid available in the area or space where the medical device 10 is operated, such as blood, urine etc., as is schematically shown in FIG. 22a, where the fluid surrounds the EAP material.

Alternatively, the electrolyte may be an ionic solution that is externally applied to the device, for instance from inside of the catheter as is illustrated in FIG. 22b.

In yet another embodiment, which is illustrated in FIG. 22c, the device may be coated with a solid electrolyte 17, such as a solid polymer electrolyte or an ion exchange coating that is soaked with an electrolyte, in a per se known manner. The solid electrolyte should be in contact with both the EAP material, the counter electrode, and the reference electrode (if any).

In one embodiment, which is illustrated in FIG. 23, the medical device 10 is used in it bare form, with the EAP exposed to the body. However, as is sketched in FIG. 23, it is possible to coat whole or a part of the medical device with a coating 18 over at least the electroactive segment, including the electrodes and electrolyte, in order to isolate the electroactive segment from the body into which it is inserted.

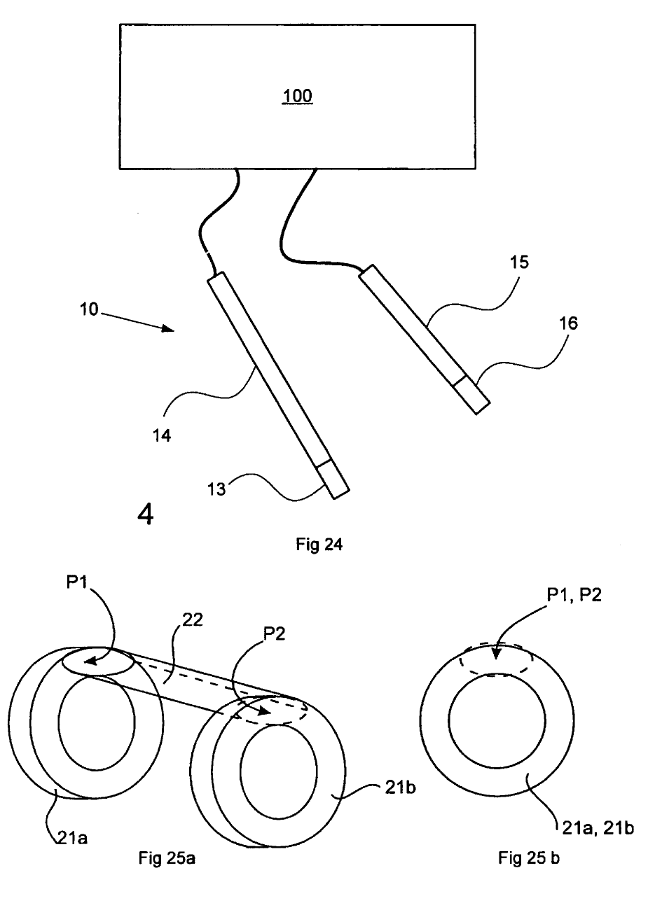

FIG. 24 schematically illustrates a system according to an aspect of the present disclosure. The system comprises a control unit 100, an elongate device 10, 30, 40, 60, 70 having a controllable 13 and a non-controllable 14 portion. The system may, where needed, comprise separate counter electrode device 15 having a counter electrode 16. The device 10 and/or the counter electrode device 15 may be connected to the control unit 100 by cables or wirelessly.

The system may be operated as follows. The elongate device 10, 30, 40, 60, 70 is introduced into the body lumen, whereby its bending or stiffness is controlled by inputting control data to the control unit 100, which in turn provides control signals to the controllable portions 13 of the elongate device 10, 30, 40, 60, 70, whereby the controllable portions bend or change their stiffness accordingly. In the case where the counter electrode 16 is not provided on the device 10, 30, 40, 60, 70, a separate counter electrode device 15 may be provided, which is also introduced in the body lumen, or in another body lumen, which is in ionic contact with the first-mentioned body lumen.

FIGS. 25a-25b illustrate in more detail a possible relationship between two adjacent skeletal members 21a, 21b and an electroactive material 22 arranged therebetween, so as to cover only a portion P1, P2 of the cross sectional area of the device.

The devices described herein may be catheters (e.g guide catheters, balloon catheter), endoscopes, guidewires, leads (such as for cardiac rhythm management, internal defibrillators, infusion), electrodes, cannulas, embolic protection devices, introduces, sheaths, etc. The device may be a device that is temporarily inserted into the body lumen during a longer or shorter time period, or a device that is (permanently) implanted into the body.

The electroactive polymer may be a conductive polymer comprising pyrrole, aniline, thiophene, para-phenylene, vinylene, and phenylene polymers and copolymers thereof, including substituted forms of the different monomers.

The devices described herein may be used as a tool carrier for such tools as are described in WO00/78222, the entire contents of which is hereby incorporated by reference. Non-limiting examples of such tools include stents, scissors, knives, balloons etc.

1. An elongate device for introduction into a body lumen, comprising:
a body extending in a longitudinal direction, and having at least two skeletal members, which are substantially aligned in the longitudinal direction, and
at least one electroactive polymer material, which changes volume upon electrical activation, arranged to control a distance between two longitudinally spaced-apart portions of said skeletal members,
whereby said body presents an asymmetric bending stiffness, and/or the electroactive polymer material is asymmetrically arranged about a central axis of the device,
such that the body is arranged to bend transversely of the longitudinal direction upon activation of the electroactive polymer material, wherein
the electroactive polymer material is form fit onto an outside of at least one of the skeletal members

2. The elongate device as claimed in claim 1, wherein the electroactive polymer material is formed directly onto said at least one of the skeletal members.

3. The elongate device as claimed in claim 1, wherein the at least one electroactive polymer material extends over a portion of a cross section in a transverse direction of the device, which portion is smaller than the total area of the device cross section.

4. The elongate device as claimed in claim 1, wherein the device further comprises:
a second electroactive polymer material arranged to control a distance between another two longitudinally spaced-apart portions of said skeletal members.

5. The elongate device as claimed in claim 1, wherein at least one of the skeletal members, has a varying thickness in a direction perpendicular to the longitudinal direction of the device.

6. The elongate device as claimed in claim 1, wherein the skeletal members form separate parts, which are arranged in a longitudinally spaced relationship to form the device.

7. The elongate device as claimed in claim 1, wherein the skeletal members are connected to each other forming substantially a helix.

8. The elongate device as claimed in claim 1, wherein longitudinally spaced-apart portions of the skeletal members are connected to each other, by a material other than the electroactive polymer material.

9. The elongate device as claimed in claim 1, wherein a material is arranged to cover at least a part of the skeletal members.

10. A method for providing an elongate device for introduction into a body lumen, comprising a body extending in a longitudinal direction, and having at least two skeletal members, which are substantially aligned in the longitudinal direction, and
at least one electroactive polymer material, which changes volume upon electrical activation, arranged to control a distance between two longitudinally spaced-apart portions of said skeletal members,
whereby said body presents an asymmetric bending stiffness, and/or the electroactive polymer material is asymmetrically arranged about a central axis of the device,
such that the body is arranged to bend transversely of the longitudinal direction upon activation of the electroactive polymer material, wherein
form-fitting the electroactive polymer material onto an outside of the skeletal members.

11. The method as claimed in claim 10, wherein the electroactive polymer material is formed directly onto at least one of the skeletal members.

12. A system comprising an elongate device for introduction into a body lumen as claimed in claim 1, and a control unit, coupled to said electroactive polymer material for providing control signals thereto.

13. The system as claimed in claim 12, further comprising a counter electrode and an electrolyte, and optionally a reference electrode.

14. The system as claimed in claim 12, wherein the electrolyte is a physiological fluid.

15. The system as claimed in claim 12, wherein the device comprises a tubular member, and wherein the electrolyte is provided inside the tubular member.
